SEQUENCE LISTING (10 sequences)

SEQ ID NO: 1 - DNA, 1728 bp, Nicotiana tabacum
  CDS (244)..(1566)
agaaacccagaaagaacaacaggttttgcttcttgttgatgagtgcatttgcctctgcttgtgtaaggcaaagtcggttcactttcttatatccgatttttataatcgttgaaattagtggatagactctagtggatatctacaagtattggttttttgataaaataggctgaggtgagaaggtaacataaaggaaagacaaaaacttgggaattgttttagaccaccgaggtttcttgtttcatgagcatgtctgttgctttgttgtgggttgtttctcccacttccgaggtctcgaatgggacaggattgttggattcagtccgagaaggaaaccgcgtctttgtatcatccaggttcctagctcgagataggaatttgatgtggaatgggagaatcaagaaaggtgggagacaaaggtggaattttggctctttaattgctgatccaagatattcatgcttgggtggatcaagaactgaaaagggaagcactttctctgtacagtccagtttggtggctagcccagctggagaaatgactgtgtcatcagagaaaaaggtgtatgatgtggtattaaagcaggcagctttagtgaagaggcagctgagatctaccgatgatttagaagtgaagccggatattgttgttccagggaatttgggcttgttgagtgaagcatatgatcgttgtggcgaagtatgtgcagagtatgcaaagacattttacttaggaaccaagctaatgaccccagagagaagaagagctatctgggcaatatatgtgtggtgcaggagaacggatgagcttgttgatggccctaatgcatcccacataactccgcaagctttagataggtgggagaccaggctggaagatattttcagtgggcggccatttgatatgcttgatgctgctttatccgatactgtctccagatttcctgttgatattcagccattcagagatatgattgaaggaatgcgtatggacttgtggaaatccagatacaaaactttcgatgagctatatctctattgttactatgttgctggtactgtaggattgatgagtgttccagttatgggtattgcacctgaatcaaaggcaacaacagagagtgtatataatgctgctttggctttagggcttgcaaatcaactaaccaatatactcagagatgtaggagaagatgccagaagaggaagagtatacttgcctcaagatgaattagcacaggcagggctctccgacgaagacatatttgctggaagagtgactgataagtggaggaactttatgaagaaacaaattcagagggcgaggaaattctttgatgagtcagagaaaggtgtcacagaactggactctgctagtagatggcctgtgttaacagcgctgctgttgtatcgcaagatattggacgagattgaagccaacgactacaacaacttcacaaggagggcttatgttagcaagccaaagaagcttctcaccttgcccattgcttatgcaaaatctcttgtgccccctaatagaacttcctctccactagcaaagacatgaatgaagtagttgagtcaatgagtattatacactaaagaaactcaggtacttgtaaatgagatatcttttgctaaatgtgtatcatcaaaagtagattgtaaattcaatatgacaatctcttggtagaatattttctccacactcatcaaaccctcaagtgag

SEQ ID NO: 2 - protein, 440 aa, Nicotiana tabacum
MSMSVALLWVVSPTSEVSNGTGLLDSVREGNRVFVSSRFLARDRNLMWNGRIKKGGRQRWNFGSLIADPRYSCLGGSRTEKGSTFSVQSSLVASPAGEMTVSSEKKVYDVVLKQAALVKRQLRSTDDLEVKPDIVVPGNLGLLSEAYDRCGEVCAEYAKTFYLGTKLMTPERRRAIWAIYVWCRRTDELVDGPNASHITPQALDRWETRLEDIFSGRPFDMLDAALSDTVSRFPVDIQPFRDMIEGMRMDLWKSRYKTFDELYLYCYYVAGTVGLMSVPVMGIAPESKATTESVYNAALALGLANQLTNILRDVGEDARRGRVYLPQDELAQAGLSDEDIFAGRVTDKWRNFMKKQIQRARKFFDESEKGVTELDSASRWPVLTALLLYRKILDEIEANDYNNFTRRAYVSKPKKLLTLPIAYAKSLVPPNRTSSPLAKT

SEQ ID NO: 3 - DNA, 1712 bp, Nicotiana tabacum
  CDS (333)..(1565)
  unsure 135, 139: Xaa is unknown or other
cttgaagagtagcagcagcaagcaagahaattaaagtgggctatttbkkanaagccattgttacmagaraattaagaagccaagamacaggttattttctacttgagtyaggaaaagttggtttgctttatttgtgggctttttataatcttttttccacaagggaaagtgggtattttcttgaaagtggatttagactctagtgggaatctactaggagtaaatttattaattttttataaattaagcagaggaaggaaggaaacagaaaacagaaagtaagacaaaaaaccttggaattgttttagaaagccaaggttttcctgttcaaaatgtctgttgccttgttatgggttgtttcaccttgtgaagtctcaaatgggacaggattcttggattcagtccgggagggaaaccgggtttttgattcgtcgaggcataggaatttagtgtgcaatgagagaaacaagagaggtgtgaaacaaaggtggaattttggttctgtaaggtctgctatggtggctacaccggcgggagaaatggcgacgatgacatcagaacagatggtttatgatgtggttttaaaacaagcagctttagtgaagaggcagttgagatctgctgatgatttagaagtgaagccggagatccctctccccgggaatttgagcttgttgagtgaagcatatgataggtgtagtgaagtatgtgcagagtatgcaaagacattttactthggaaccatgytaatgactccagagagaagaagggctatttgggcaatatatgtgtggtgcaggagaacagatgaacttgttgatggcccaaacgcatcacatattacaccccaagccttagataggtgggaagaccggcttgaagatgttttcagcgggcgaccatttgatatgctcgatgctgctttgtccgatactgtttccaagtttccagttgatattcagccgttcagagatatgattgaaggaatgcgtatggacttgaggaagtcaagatatagaaactttgatgagctttacctctattgttattacgttgctggtacggttgggttgatgagtgttccaattatgggtattgcacctgattcaaaggcaacaacagagagcgtatataatgcagctttggctttaggaatcgcaaatcaactaacgaacatactcagagatgttggagaagatgccagaagaggaagagtctacttacctcaagatgaattagcacaggcaggtctcttcgacgatgacatatttgctggaaaagtgactgataagtggagaagctttatgaagaagcaaatccagagggcaagaaagttcttcgatgaggcagaggaaggagttacacaactgagctcagctagcagatggcctgtatgggcatctttgctgttgtaccgccaaatactggacgagattgaagccaatgactacaacaacttcacaaagagagcttatgtgagcaaaccaaagaagctaatttccttacctattgcttatgcaaaatctcttgtgccccctacaagaactcttgtcacctctagctaaggcatagacatcagatttaaattaaagcaagaaagcatatactgttaaaaaagaaagaatttctaaagtagatattgttgtattgatgccacttgtatatcatcaaaagtaggtagtaaaatccaatataacaatctctagtagttg

SEQ ID NO: 4 - protein, 410 aa, Nicotiana tabacum
  unsure 135, 139: Xaa is unknown or other
MSVALLWVVSPCEVSNGTGFLDSVREGNRVFDSSRHRNLVCNERNKRGVKQRWNFGSVRSAMVATPAGEMATMTSEQMVYDVVLKQAALVKRQLRSADDLEVKPEIPLPGNLSLLSEAYDRCSEVCAEYAKTFYXGTMXMTPERRRAIWAIYVWCRRTDELVDGPNASHITPQALDRWEDRLEDVFSGRPFDMLDAALSDTVSKFPVDIQPFRDMIEGMRMDLRKSRYRNFDELYLYCYYVAGTVGLMSVPIMGIAPDSKATTESVYNAALALGIANQLTNILRDVGEDARRGRVYLPQDELAQAGLFDDDIFAGKVTDKWRSFMKKQIQRARKFFDEAEEGVTQLSSASRWPVWASLLLYRQILDEIEANDYNNFTKRAYVSKPKKLISLPIAYAKSLVPPTRTLVTSS

SEQ ID NO: 5 - DNA, 2205 bp, Nicotiana tabacum
  CDS (189)..(1955)
ctggcatcttacatctgccaaatttctcatttatagcatctcctaatctttagataccttttcttcttgttttgtttttctatccttcacttcatgctttcttgttttacccatctcttccattttcttggcatttgacaacaaaaggttccattttttttcctttttgctgtatatagcacaattcaatggctacttcttcagcttatctttgttgtcctgcaacttctgctactggaaagaaacatattttgccaaatgggtcagctggattcttggttttccgtggtccccgtttgtccaaccggtttgtgacccggaagtcagttattcgtgctgatttggactccatggtctctgatatgagtactaatgctccaaaagggctatttccacctgaacctgaacattatcgggggccaaagctgaaagtagctattattggagctgggcttgcaggcatgtcaactgctgtggagctcttggatcaaggacatgaggtggatatatatgaatcaaggccttttattggtgggaaagtgggatcttttgttgatagacgtggaaaccacattgaaatgggactgcatgtgttctttggttgctataataatttgttccgtttgttaaaaaaggtgggtgctgaaaaaaatctgctagtgaaggaccatactcacacatttgtaaataaagggggtgaaataggggagcttgatttccgctttccagttggagcacccctacacggaattaatgcatttttgtctaccaatcagctaaagatttatgataaggctagaaatgctgtagctcttgcccttagtccagtggtgcgggctttagttgatccagatggcgcgttgcagcagatacgtgatctagatagtgtaagcttttcagagtggtttatgtctaaaggtgggacgcgtgctagcatccagaggatgtgggatcctgtcgcatatgctcttggattcattgactgtgacaatatcagtgctcggtgtatgctcactatatttgcattatttgccactaaaacggaggcttccctattacgcatgcttaaaggttctccggacgtttatttgagtggtccaattaagaagtacatcttggataaggggggaaggtttcacatgaggtgggggtgcagacaggtactctatgagacatcctctgatggcagtatgtatgtcagcgggcttgccatgtcaaaggccactcagaagaaagttgtaaaagctgatgcctatgtcgctgcatgtgatgtccctggaattaaacgattggtacctcagaagtggagggaattggaattctttgacaacatttacaaattggttggagtgcctgttgttacggtacaactacgatacaatggctgggttacagagttgcaggacttggagcgttcgaggcaattgaagcgcgctacaggtttggacaatctcctgtatacaccagatgcagatttctcttgctttgcggaccttgcattggcatctcctgaagattattacattgagggccaaggctcattgcttcaatgtgtccttacacctggtgacccttacatgcctctactaaatgatgaaatcataaaaagagtgtcaaagcaggttttggcactatttccttcttcccaaggtcttgaggttacctggtcatcagttgtgaaaattgggcaatccctatatcgtgaaggacctggtaaagacccattcagacctgatcagaagactccagtggaaaatttctttcttgctggctcatatacaaaacaggactacatagatagcatggaaggggcaactctttcaggtaggcaagcatctgcatacgtatgtgatgctggcgagaagctggtggtgttgcggaaaaagattgctgctgctgagtcaaacgagatctctgaaggtgtatcagtatctgatgagttgagtcttgtctgatgactggaaatcatccaatgaatactgaagagcaccccccactttgttaatccgagaagcagatacaaacataactcagttaggcattgcgtaaggaagagttcttctaaattttgagttcacaagatggaaatcaaaaggttaaaatatgttgtatgtaatattagtaaatcttcatagtgatgtatctattctgccacccttcaggtttagtgaaatggatcgtattgctcatcattcattgagaaga

SEQ ID NO: 6 - protein, 588 aa, Nicotiana tabacum
MATSSAYLCCPATSATGKKHILPNGSAGFLVFRGPRLSNRFVTRKSVIRADLDSMVSDMSTNAPKGLFPPEPEHYRGPKLKVAIIGAGLAGMSTAVELLDQGHEVDIYESRPFIGGKVGSFVDRRGNHIEMGLHVFFGCYNNLFRLLKKVGAEKNLLVKDHTHTFVNKGGEIGELDFRFPVGAPLHGINAFLSTNQLKIYDKARNAVALALSPVVRALVDPDGALQQIRDLDSVSFSEWFMSKGGTRASIQRMWDPVAYALGFIDCDNISARCMLTIFALFATKTEASLLRMLKGSPDVYLSGPIKKYILDKGGRFHMRWGCRQVLYETSSDGSMYVSGLAMSKATQKKVVKADAYVAACDVPGIKRLVPQKWRELEFFDNIYKLVGVPVVTVQLRYNGWVTELQDLERSRQLKRATGLDNLLYTPDADFSCFADLALASPEDYYIEGQGSLLQCVLTPGDPYMPLLNDEIIKRVSKQVLALFPSSQGLEVTWSSVVKIGQSLYREGPGKDPFRPDQKTPVENFFLAGSYTKQDYIDSMEGATLSGRQASAYVCDAGEKLVVLRKKIAAAESNEISEGVSVSDELSLV

SEQ ID NO: 7 - DNA, 24 bp, Nicotiana tabacum
aacccgggatagcacgattcaatg

SEQ ID NO: 8 - DNA, 25 bp, Nicotiana tabacum
aacccgggatttccagtcatcagac

SEQ ID NO: 9 - DNA, 22 bp, Nicotiana tabacum
ttcccgggctcagtaaaatgcc

SEQ ID NO: 10 - DNA, 23 bp, Nicotiana tabacum
tacccgggctaaactacgcttgc

(19) United States
(12) Patent Application Publication
(10) Pub. No.: US 2002/0128464 A1
(43) Pub. Date: Sep. 12, 2002

(54) Method of finding modulators of enzymes of the carotenoid biosynthetic pathway

(76) Inventors: Marco Busch, Langenfeld (DE); Rudiger Hain, Langenfeld (DE)

Correspondence Address: BAYER CORPORATION, PATENT DEPARTMENT, 100 BAYER ROAD, PITTSBURGH, PA, 15205, US

(21) Appl. No.: 09/847,081

(22) Filed: May 2, 2001

(30) Foreign Application Priority Data
May 8, 2000 (DE) 10022362.1

(51) Int. Cl. C07H 21/04; C12N 9/02; A01H 5/00

(52) U.S. Cl. 536/23.6; 435/189; 435/410; 800/317.3

(57) ABSTRACT

The invention relates to nucleic acids which encode tobacco zeta-carotene synthase, to polypeptides encoded thereby, and to methods of finding modulators of the activity of zeta-carotene synthase, phytoene synthase and phytoene desaturase.

The invention relates to nucleic acids which encode tobacco zeta-carotene synthase, to polypeptides encoded thereby, and to methods of finding modulators of the activity of zeta-carotene synthase, phytoene synthase and phytoene desaturase.

Undesired plant growth can be prevented by the use of herbicides. The demands made on herbicides have increased constantly with regard to their efficacy, costs and environmental compatibility. There is therefore a need for new substances which can be developed into potent new herbicides. In general, the usual procedure is to search for such new lead structures in greenhouse tests. However, such tests are laborious and expensive. Accordingly, the number of substances which can be tested in the greenhouse is limited.

Advantageous herbicide targets are being searched for in plant-specific biosynthetic pathways which do not occur in animal organisms. One example is the carotenoid biosynthetic pathway.

The carotenoids play a number of roles in plant metabolism. In the photosynthetic system, they are associated with the light harvesting complex, which guarantees the optimal transmission of the incident photons to the photosynthetic reaction centres. Furthermore, they participate in dissipation of excess light energy and the scavenging of free oxygen radicals and, accordingly, have a protective function. In addition to their importance for photosynthesis, the carotenoids are precursors for the biosynthesis of the xantophylls and the growth regulator abscisic acid. In flowers and fruits, the carotenoids act as pigments, for example lycopene in tomato (Lycopersicon esculentum) or β-carotene in carrott (Daucus carota).

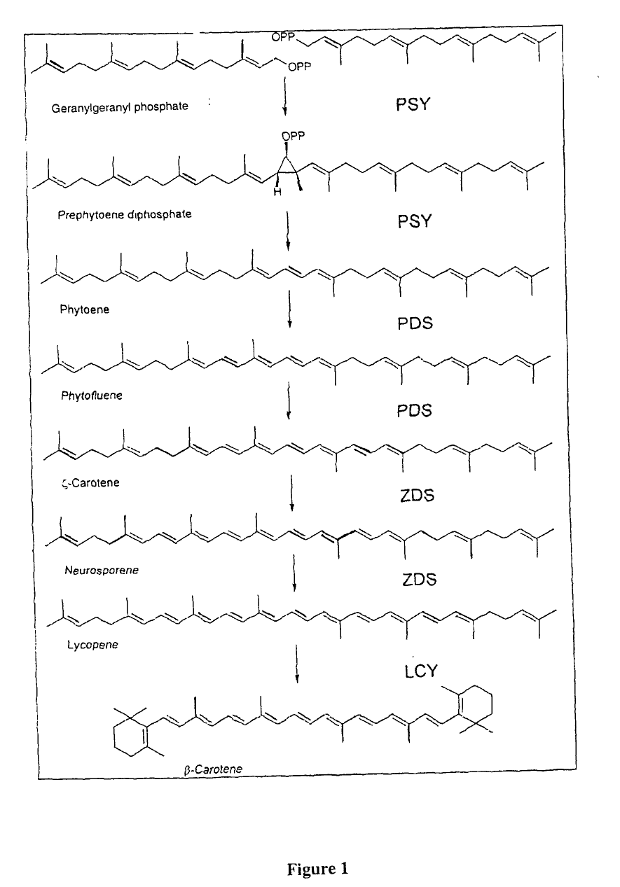

Carotenoid biosynthesis takes place in the plastids. The precursor for the synthesis is the diterpene geranylgeranyl pyrophosphate. It is probably formed via the so-called DXP or else Rohmer pathway and not via the standard mevalonate pathway (Lichtenthaler, 1997). Two molecules of geranylgeranyl pyrophosphate (GGPP) are converted by phytoene synthase (PSY) by head-to-head coupling to give one molecule of phytoene (FIG. 1). The enzymes phytoene desaturase (PDS) and zeta-carotene desaturase (ZDS) successively introduce four further double bonds. Each enzyme catalyses dehydrogenation at symmetric positions (FIG. 1).

The lycopene which forms is then converted in a first step either by lycopene ε-cyclase (LCYe) to give δ-carotene or by lycopene β-cyclase (LCYb) to give γ-carotene. In a second step, this gives α- and β-carotene, respectively. This second cyclization step is performed in each case by lycopene β-cyclase. α- and β-carotene then constitute the precursors for the synthesis of xantophylls such as lutein, astaxanthin, violaxanthin and the like.

All enzymes which participate in carotenoid and xanthophyll biosynthesis are encoded by the nuclease. The gene sequences for almost all proteins are known from at least one plant. The translated preprotein is imported into the chloroplasts, processed, oligomerized and, if appropriate, translocated by a recognition sequence. The chaperone Cpn60 and the heat shock protein Hsp70 participate in this process (Bonk, 1997).

Phytoene synthase is a protein of ˜45 kDa. PSY requires manganese ions and ATP as cofactors (Dogbo, 1988). Moreover, association with galactolipids is required for the activity of the plant enzyme. When Erwinia uredovora PSY is overexpressed, a sensitivity to phosphate and a capability of being inhibited by squalestatin with a pI valve of 15 μM were measured (Neudert, 1998). This correlates with the homology of the known PSY genes of ˜34% at the amino acid level with squalene synthases and suggests that these two enzymes are closely related. Squalene synthesis proceeds via the analogous head-to-head coupling of two molecules of farnesyl pyrophosphate, which, in mechanistic terms, corresponds to the synthesis of phytoene. Light-dependent induction of expression was demonstrated for Capsicum aniuum phytoene synthase (Lintig, 1997). Overexpression of the fruit-specific PSY1 in tomato plants had the phenotypic result of dwarfism. This was attributed to geranylgeranyl pyrophosphate being redirected from gibberellin biosynthesis to carotenoid biosynthesis (Fray, 1995). DNA encoding for PSY, for example from melon and Nicotiana species, has already been described (WO 96/02650, U.S. Pat. No. 5,705,624). However, nothing has become known about the importance of phytoene synthase for plant vitality. Whether switching off the gene encoding PSY is lethal for a plant, that is to say whether the enzyme is suitable as target molecule for herbicidally active substances, has not been disclosed as yet.

Phytoene desaturase is a protein of ˜64 kDa. PDS is activated by flavinylation and utilizes plastoquinone as electron acceptor (Norris, 1995). There exists contradictory information on the regulation of phytoene desaturase. On the one hand, it has been reported that PDS gene expression is affected by the chlorophyll and pigment content (Corona, 1996) while, on the other hand, dependence of PDS expression on the pigment content is denied (Woetzel, 1998). Following inhibition of PDS with the known inhibitors norflurazon and fluridone, a loss of photosystem II activity was detected in vitro. This loss of activity was attributed to the necessity of the presence of β-carotene for D1 protein to be incorporated into a functional photosystem II (Trebst, 1997). However, whether switching off phytoene desaturase is lethal for the plant, that is to say whether the enzyme is suitable as target molecule for herbicidal active substances, has not been studied as yet. The sequence of the Nicotiana tabacum phytoene desaturase is described in document U.S. Pat. No. 5,539,093. This sequence is expressly intended to be part of the present application. DNA sequences encoding PDS can be found, inter alia, in WO 99/55888.

Zeta-carotene desaturase has a size of ˜65 kDa and is the least characterized enzyme of carotenoid biosynthesis. The sequences of the known plant ZDSs, for example from rice, maize, wheat, soya or Capsicum anuum (WO 99/55888), show homologies around 34% with the known PDS sequences. No information exists as yet on ZDS regulation.

Lycopene β-cyclase is a protein with a size of ˜55 kDa. In the plastids, it competes with lycopene ε-cyclase for lycopene, which they share as substrate. In contrast to the β-cyclases, whose genes are known from a variety of plants, only the plant genes of Arabidoposis thaliana and tomato are known in the case of ε-cyclase. The comparison of the sequences of the two cyclase types shows a homology of ˜36% at the amino acid level. Lycopene cyclization by the two cyclases constitutes a branching point in carotenoid biosynthesis and thus a meaningful point of regulation. The ratio between β- and ε-cyclase increases under strong light, and more of the protective xantophylls zeaxanthin, violaxanthin and antheraxanthin are formed. In weak light, the ratio of β- to ε-cyclase decreases, and more lutein, which participates in light harvesting, is formed (Cunningham, 1996).

Carotenoid biosynthesis and xanthophyll biosynthesis are highly regulated processes. Biosynthesis is regulated in the chloroplasts of the photosynthetic tissue as a function of light intensity. In contrast, regulation in the chromoplasts of the flowers and fruits depends on the developmental stage. In tomato fruits (Lycopersicon esculentum), the red coloration during maturation is achieved by the accumulation of lycopene. This accumulation goes hand in hand with increased quantities of PSY and PDS transcripts. At the same time, the transcripts for the lycopene cyclases disappear (Pecker, 1996).

Only little is known on the details of the mechanisms by which carotenoid biosynthesis is regulated.

The present application describes the cloning of genes of carotenoid biosynthesis. Inter alia, two phytoene synthase genes have been found. It was not possible to demonstrate an analogy to development-dependent regulation, as in the case of tomato. There is the possibility of light intensity-dependent regulation. In this case, one gene might encode the housekeeping activity while the other might be regulated in a light intensity-dependent fashion.

The present application also describes the cloning of the gene encoding zeta-carotene desaturase.

The application also describes the cloning of the gene encoding the Nicotiana tabacum phytoene desaturase.

The present application also describes that the known enzymes of the carotenoid biosynthetic pathway, namely phytoene synthase, phytoene desaturase and zeta-carotene desaturase, which are connected to each other owing to the catalysis of consecutive steps in carotenoid biosynthesis, are of essential importance in plants. The present application also describes that the enzymes phytoene synthase, phytoene desaturase and zeta-carotene desaturase are suitable as target molecules for herbicidal active substances and can therefore be used in methods of finding herbicidal active substances.

Accordingly, the present invention relates to the use of the enzymes of carotenoid biosynthesis, namely phytoene synthase, phytoene desaturase and zeta-carotene desaturase, in methods of finding herbicidally active substances.

Accordingly, the present invention relates to nucleic acids which encode plant polypeptides with the bioactivity of a phytoene synthase, which comprises the amino acid sequence of SEQ ID NO. 2 or SEQ ID NO. 4. In particular, the nucleic acids according to the invention encode tobacco phytoene synthase, the Nicotiana tabacum SR1 nucleic acids according to the invention being especially preferred.

The present invention also relates to fragments of the nucleic acids according to the invention which encode phytoene synthase.

The present invention also relates to nucleic acids which encode plant polypeptides with the bioactivity of a zeta-carotene desaturase, which comprises the amino acid sequence of SEQ ID NO. 6. In particular, the nucleic acids according to the invention encode tobacco zeta-carotene desaturase, the Nicotiana tabacum SR1 nucleic acids according to the invention being especially preferred.

The present invention also relates to fragments of the nucleic acids according to the invention which encode zeta-carotene desaturase.

The nucleic acids according to the invention are, in particular, single-stranded or double-stranded deoxyribonucleic acids (DNAs) or ribonucleic acids (RNAs). Preferred embodiments are fragments of genomic DNA which, if appropriate, may also contain introns, and cDNAs.

The fragments may also be single-stranded or double-stranded, it being possible for single-stranded fragments to be complementary to the codogenic or to the coding strand of the nucleic acids according to the invention. Such single-stranded fragments can then hybridize either with the codogenic or the coding strand of the nucleic acid according to the invention.

The term “fragment” as used in the present context comprises single-stranded or double-stranded nucleic acids with a length of 10 to 1000 base pairs (bp), preferably with a length of 12 to 500 bp, especially preferably with a length of 15 to 200 bp, and very especially preferably with a length of 20 to 100 base pairs.

The nucleic acids according to the invention are preferably DNA which corresponds to the genomic DNA of tobacco plants which may contain introns, or fragments thereof.

The nucleic acids according to the invention especially preferably comprise a sequence selected from amongst

a) the sequence of SEQ ID NO: 1, 3 or 5,

b) sequences encoding a polypeptide which comprises the amino acid sequence

of SEQ ID NO: 2, 4 or 6,

c) part sequences of the sequences defined under a) or b) which are at least 14 base pairs in length,

d) sequences which hybridize with the sequences defined under a), b) or c),

e) sequences which are complementary to the sequences defined under a), b) or c), and

f) sequences which, owing to the degeneracy of the genetic code, encode the same amino acid sequence as the sequences defined under a) to c).

A very especially preferred embodiment of the nucleic acids according to the invention is a cDNA molecule with the sequence of SEQ ID NO: 1.

Another very especially preferred embodiment of the nucleic acids according to the invention is a cDNA molecule with the sequence of SEQ ID NO: 3.

Another very especially preferred embodiment of the nucleic acids according to the invention is a cDNA molecule with the sequence of SEQ ID NO: 5.

The term “to hybridize” as used in the present context describes the process in which a single-stranded nucleic acid molecule undergoes base pairing with a complementary strand. Starting from the sequence information disclosed herein, it is possible, in this manner, to isolate from plants other than tobacco plants for example DNA fragments which encode phytoene synthase or zeta-carotene desaturase and which have the same or similar properties as the enzymes with the amino acid sequence of SEQ ID NO: 2 or 4, or SEQ ID NO: 5.

Hybridization conditions are calculated by approximation using the following formula:

The melt temperature Tm=81.5° C.+16.6 log{c(Na⁺)]+0.41(% G+C))−500/n (Lottspeich and Zorbas, 1998).

In this formula, c is the concentration and n the length of the hybridizing sequence segment in base pairs. For a sequence >100 bp, the term 500/n is omitted. Highest stringency means washing at a temperature of 5-15° C. below Tm and an ionic strength of 15 mM Na⁺ (corresponds to 0.1×SSC). If an RNA sample is used for hybridization, the melting point is 10 to 15° C. higher.

Preferred hybridization conditions are stated below:

Hybridization solution: 6×SSC/5×Denhardt's solution/50% formamide;

Hybridization temperature: 36° C., preferably 42° C.;

Wash step 1: 2×SSC, 30 minutes at room temperature;

Wash step 2: 1×SSC, 30 minutes at 50° C.; preferably 0.5×SSC, 30 minutes at 65° C.; especially preferably 0.2×SSC, 30 minutes at 65° C.

The degree of nucleic acid identity is preferably determined with the aid of the programme NCBI BLASTN Version 2.0.4. (Altschul et al., 1997).

The present invention also relates to the regulatory regions which naturally control, in plant cells, in particular in tobacco plants, the transcription of the nucleic acids according to the invention.

The term “regulatory regions” as used in the present context relates to untranslated regions of the gene in question, such as promoters, enhancers, repressor or activator binding sites, or termination sequences which interact with cellular proteins, thus controlling transcription.

The present invention furthermore relates to the DNA constructs comprising a nucleic acid according to the invention and a heterologous promoter.

The term “heterologous promoter” as used in the present context relates to a promoter which has properties other than the promoter which controls the expression of the gene in question in the original organism.

The choice of heterologous promoters depends on whether pro- or eukaryotic cells or cell-free systems are used for expression. Examples of heterologous promoters are the cauliflower mosaic virus 35S promoter for plant cells, the alcohol dehydrogenase promoter for yeast cells, the T3, T7 or SP6 promoters for prokaryotic cells or cell-free systems.

The present invention furthermore relates to vectors which contain a nucleic acid according to the invention, a regulatory region according to the invention or a DNA construct according to the invention. Vectors which can be used are all phages, plasmids, phagemids, phasmids, cosmids, YACs, BACs, artificial chromosomes or particles which are suitable for particle bombardment, all of which are used in molecular biology laboratories.

Preferred vectors are pBIN (Bevan, 1984) and its derivatives for plant cells, pFL61 (Minet et al., 1992) for yeast cells, pBLUESCRIPT vectors for bacterial cells, and lamdaZAP (Stratagene) for phages.

The present invention also relates to host cells which contain a nucleic acid according to the invention, a DNA construct according to the invention or a vector according to the invention.

The term “host cell” as used in the present context refers to cells which do not naturally contain the nucleic acids according to the invention.

Suitable host cells are not only prokaryotic cells, preferably E. coli, but also eukaryotic cells, such as cells of Saccharomyces cerevisiae, Pichia pastoris, insects, plants, frog oocytes and mammalian cell lines.

The present invention furthermore relates to polypeptides with the bioactivity of a phytoene synthase which are encoded by the nucleic acids according to the invention. They are, in particular, polypeptides which constitute phytoene synthases according to the invention. The present invention very particularly relates to polypeptides which correspond to an amino acid sequence of SEQ ID NO: 2 or SEQ ID NO: 4.

The present invention furthermore relates to polypeptides with the bioactivity of a zeta-carotene desaturase which are encoded by the nucleic acids according to the invention. They are, in particular, polypeptides which constitute zeta-carotene desaturases according to the invention. The present invention very particularly relates to polypeptides which correspond to an amino acid sequence of SEQ ID NO: 6.

The term “polypeptide” as used in the present context refers not only to short amino acid chains which are usually termed peptides, oligopeptides or oligomers, but also longer amino acid chains which are usually termed proteins. It comprises amino acid chains which can be modified either by natural processes, such as post-translational processing, or by state-of-the-art chemical processes. Such modifications can occur at different sites and repeatedly in a polypeptide, such as, for example, at the peptide backbone, at the amino acid side chain, at the amino terminus and/or at the carboxyl terminus. They comprise, for example, acetylations, acylations, ADP-ribosylations, amidations, covalent linkages with flavins, haem constituents, nucleotides or nucleotide derivatives, lipids or lipid derivatives or phosphatidylinositol, cyclizations, formations of disulphide bridges, demethylations, cystin formations, formylations, gamma-carboxylations, glycosylations, hydroxylations, iodinations, methylations, myristoylations, oxidations, proteolytic processings, phosphorylations, selenoylations and tRNA-mediated additions of amino acids.

The polypeptides according to the invention may exist in the form of “mature” proteins or as parts of larger proteins, for example as fusion proteins. They may furthermore exhibit secretion or leader sequences, pro-sequenes, sequences which make possible simple purification, such as multiple histidine residues, or additional stabilizing amino acids.

The polypeptides according to the invention need not represent a complete phytoene synthase or zeta-carotene desaturase, but may also just be fragments thereof as long as they retain at least one bioactivity of the complete phytoene synthase or zeta-carotene desaturase. Such fragments, which exert a bioactivity of the same kind as a phytoene synthase with an amino acid sequence of SEQ ID NO: 2 or SEQ ID NO: 4 or a bioactivity of the same kind as a zeta-carotene desaturase with an amino acid sequence of SEQ ID NO: 6, are considered to be in accordance with the invention.

In comparison with the corresponding regions of naturally occurring tobacco phytoene synthases or zeta-carotene desaturases, the polypeptides according to the invention may exhibit deletions or amino acid substitutions as long as they exert at least one bioactivity of the complete enzymes. Conservative substitutions are preferred. Such conservative substitutions comprise variations, where amino acid is replaced by another amino acid amongst the following group:

1. small aliphatic residues, nonpolar residues or residues of little polarity: Ala, Ser, Thr, Pro and Gly;

2. polar, negatively charged residues and their amides: Asp, Asn, Glu and Gln;

3. polar, positively charged residues: His, Arg and Lys;

4. large aliphatic unpolar residues: Met, Leu, Ile, Val and Cys; and

5. aromatic residues: Phe, Tyr and Trp.

Preferred conservative substitutions can be seen from the following list:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

A preferred embodiment of the polypeptides according to the invention is the tobacco phytoene synthase with the amino acid sequence of SEQ ID NO: 2 or the amino acid sequence of SEQ ID NO: 4.

A further preferred embodiment of the polypeptides according to the invention is the tobacco zeta-carotene desaturase with the amino acid sequence of SEQ ID NO: 6.

The present invention furthermore relates to antibodies which bind specifically to the polypeptides according to the invention. The generation of such antibodies follows customary procedures. These antibodies can be utilized for example for identifying expression clones, for example of a genetic library, which carry the nucleic acids according to the invention.

The term “antibody” as used in the present context also extends to parts of complete antibodies such as Fa, F(ab′)₂ or Fv fragments, which are still capable of binding to the epitopes of the polypeptides according to the invention.

The present invention also relates to processes for generating the nucleic acids according to the invention. The nucleic acids according to the invention can be generated in the customary manner. For example, the nucleic acid molecules can be synthesized completely by chemical synthesis. It is also possible to chemically synthesize short segments of the nucleic acids according to the invention and to radiolabel such oligonucleotides or to label such oligonucleotides with a fluorescent dye. The labelled oligonucleotides can also be used to screen cDNA libraries made with plant mRNA as starting material. Clones which hybridize to the labelled oligonucleotides are selected for isolating the DNA fragments in question. After the DNA isolated has been characterized, the nucleic acids according to the invention are obtained in a simple manner.

The nucleic acids according to the invention can also be generated by means of PCR methods using chemically synthesized oligonucleotides.

The term “oligonucleotide(s)” as used in the present context denotes DNA molecules consisting of 10 to 50 nucleotides, preferably 15 to 30 nucleotides. They are synthesized chemically and can be used as probes.

The present invention furthermore relates to processes for generating the polypeptides according to the invention. To generate the polypeptides which are encoded by the nucleic acids according to the invention, host cells which contain nucleic acids according to the invention may be cultured under suitable conditions. Thereafter, the desired polypeptides can be isolated in the customary manner from the cells or the culture medium. The polypeptides may also be generated in in-vitro systems.

A rapid method of isolating the polypeptides according to the invention, which are synthesized by host cells using a nucleic acid according to the invention, starts with expressing a fusion protein, it being possible for the fusion partner to be affinity-purified in a simple manner. The fusion partner may be, for example, glutathion S transferase. The fusion protein can then be purified on a glutathion affinity column. The fusion partner can be separated by partial proteolytic cleavage for example at linkers between the fusion partner and the polypeptide according to the invention which is to be purified. The linker can be designed such that it includes target amino acids, such as arginine and lysine residues, which define sites for trypsin cleavage. In order to generate such linkers, standard cloning methods using oligonucleotides may be used.

Further purification processes which are possible are based on preparative electrophoresis, FPLC, HPLC (for example using gel filtration, reversed-phase or mildly hydrophobic columns), gel filtration, differential precipitation, ion-exchange chromatography and affinity chromatography.

The terms “isolation or purification” as used in the present context denote that the polypeptides according to the invention are separated from other proteins or other macromolecules of the cell or of the tissue. A composition containing the polypeptides according to the invention is preferably at least 10-fold and especially preferably at least 100-fold more concentrated with regard to its protein content compared with a host cell preparation.

The polypeptides according to the invention may also be affinity-purified without fusion partner with the aid of antibodies which bind to the polypeptides.

The present invention also relates to methods of finding chemical compounds which bind to the polypeptides according to the invention and modify their properties. Such compounds can act as modulators of the polypeptides according to the invention, either as agonists or antagonists.

The present invention also relates to methods of finding chemical compounds which bind to phytoene desaturase and modify its properties, it being possible for these compounds to act as agonists or antagonists.

The term “agonist” as used in the present context refers to a molecule which accelerates or enhances the enzymatic activity of the enzyme phytoene synthase, the enzyme phytoene desaturase or the enzyme zeta-carotene desaturase.

The term “antagonist” as used in the present context refers to a molecule which slows down or inhibits the enzymatic activity of the enzyme phytoene synthase, the enzyme phytoene desaturase or the enzyme zeta-carotene desaturase.

The term “modulator” as used in the present context constitutes the generic term for agonist or antagonist. Modulators can be small organochemical molecules, peptides or antibodies which bind to the polypeptides according to the invention. Further modulators may be small organochemical molecules, peptides or antibodies which bind to a molecule which, in turn, binds to the polypeptides according to the invention, thus influencing their bioactivity. Modulators may be natural substrates and ligands or structural or functional mimetics thereof.

The modulators are preferably small organochemical compounds.

The binding of the modulators to the enzymes phytoene synthase, phytoene desaturase and zeta-carotene desaturase can alter the cellular procedures in a manner which leads to the death of the plants treated therewith.

The present invention therefore also comprises the use of the polypeptides phytoene synthase, zeta-carotene desaturase and phytoene desaturase according to the invention in methods of finding compounds which influence enzyme activity.

The present invention furthermore comprises methods of finding chemical compounds which modify the expression of phytoene synthase, zeta-carotene desaturase and phytoene desaturase. Such “expression modulators” may also constitute new growth-regulatory or herbicidal active substances. Expression modulators can be small organochemical molecules, peptides or antibodies which bind to the regulatory regions of the nucleic acids encoding phytoene synthase, zeta-carotene desaturase or phytoene desaturase. Furthermore, expression modulators may be small organochemical molecules, peptides or antibodies which bind to a molecule which, in turn, binds to regulatory regions of the nucleic acids encoding phytoene synthase, zeta-carotene desaturase or phytoene desaturase, thus influencing their expression. Expression modulators may also be antisense molecules.

The present invention therefore also extends to the use of modulators of phytoene synthase, zeta-carotene desaturase and phytoene desaturase or of expression modulators as plant growth regulators or herbicides.

The methods according to the invention include high throughput screening (HTS). Not only host cells, but also cell-free preparations which contain the nucleic acids according to the invention and/or phytoene synthase, zeta-carotene desaturase or phytoene desaturase or nucleic acids encoding them may be used for this purpose.

To find modulators, a synthetic reaction mix (for example products of the in vitro transcription) or a cellular component which contains phytoene synthase, zeta-carotene desaturase or phytoene desaturase can be incubated together with a labelled substrate or ligands of the polypeptides in the presence and absence of a candidate molecule, which may be an agonist or antagonist. The ability of the candidate molecule to increase or inhibit the activity of phytoene synthase, zeta-carotene desaturase or phytoene desaturase can be seen from an increased or reduced binding of the labelled ligand or an increased or reduced conversion of the labelled substrate. Molecules which bind well and lead to an increased activity of phytoene synthase, zeta-carotene desaturase or phytoene desaturase are agonists. Molecules which bind well but do not trigger the bioactivity of phytoene synthase, zeta-carotene desaturase or phytoene desaturase are probably good antagonists. Detection of the bioactivity of phytoene synthase, zeta-carotene desaturase or phytoene desaturase can be improved by a so-called reporter system. Reporter systems in this regard comprise, but are not limited to, colorimetrically labelled substrates which are converted into a product, or a reporter gene which responds to changes in the activity or the expression of phytoene synthase, zeta-carotene desaturase or phytoene desaturase, or other known binding tests.

A further example of a method with the aid of which modulators of phytoene desaturase and/or the polypeptides according to the invention can be found is a displacement test, in which phytoene synthase, zeta-carotene desaturase or phytoene desaturase and a potential modulator are combined under suitable conditions with a molecule which is known to bind to the polypeptides according to the invention, such as a natural substrate or ligand or a substrate or ligand mimetic. The polypeptides according to the invention or phytoene desaturase itself can be labelled, for example radiolabelled or colorimetrically labelled, so that the number of polypeptides which are bound to a ligand or which have undergone conversion can be determined accurately. This makes it possible to determine the efficacy of an agonist or antagonist.

The invention furthermore relates to the use of a nucleic acid according to the invention, of a DNA construct according to the invention or of a vector according to the invention for the generation of transgenic plants, and to the corresponding transgenic plants as such or their parts or propagation material.

Transgenic plants, parts of plants, protoplasts, plant tissues or plant propagation materials, in which the intracellular concentration of the receptor-like protein kinases is increased or reduced in comparison with the corresponding wild-type forms after introducing a nucleic acid according to the invention, a DNA construct according to the invention or a vector according to the invention, are also subject-matter of the present invention.

The term “parts of plants” as used in the present context denotes all aerial and subterranian parts and organs of the plants, such as shoot, leaf, flower and root, and protoplasts and tissue cultures made therewith.

The term “propagation material” as used in the present context denotes vegetative and generative propagation material such as cuttings, tubers, rhizomes, layers and seeds.

The invention also relates to plants in which modifications in the sequence encoding phytoene synthase are being carried out and the plants which lead to the production of a phytoene synthase according to the invention are then selected, or in which an increased or reduced endogenous phytoene synthase activity is achieved by mutagenesis.

The invention also relates to plants in which modifications in the sequence encoding zeta-carotene desaturase are being carried out and the plants which lead to the production of a zeta-carotene desaturase according to the invention are then selected, or in which an increased or reduced endogenous zeta-carotene desaturase activity is achieved by mutagenesis.

### EXAMPLES

### Example 1

### Cloning Tobacco Phytoene Synthase Genes

The 5′ and 3′ termini of PSY were cloned via RACE-PCR. The fragments obtained were sequenced. The complete sequence for PSY was put together from the sequences of the fragments. From the fragments which have been sequenced, in each case a middle and a 3′-terminal fragment showed a deviating sequence with a homology with the remaining fragments of approximately 80% at the nucleotide level. These two fragments showed 100% identity in the overlapping region. The fact that the two deviating fragments from different PCR reactions agree implied the presence of a further PSY gene in tobacco. The PSY sequence which was already complete was therefore given the suffix PSY1, the deviating sequence was given the suffix PSY2. In accordance with the partial sequence of PSY2, a new primer was defined for the RACE amplification of the corresponding 5′ terminus. 5′-Terminal fragments were obtained using this primer. The sequences of these fragments were determined. They showed 100% homology with the existing sequence of PSY2 in the overlapping region. The complete sequence of PSY2 was put together from the sequences of the fragments. The protein sequences were determined on the basis of the sequenes obtained. The open reading frame found for PSY1 encodes a protein of 439 amino acids corresponding to ˜48 kDa. The open reading frame for PSY2 encodes a protein of 410 amino acids corresponding to ˜45 kDa. The two tobacco PSY genes show 86% homology at the amino acid level. The greatest deviations are located in two deletions of 4 and 22 amino acids in the N-terminal region. At the amino acid level, PSY1 and PSY2 show 96% and 93% homology, respectively, with tomato PSY2 and 85% and 87%, respectively, with tomato PSY1. Not more than one PSY gene is known from all other plants.

The detailed cloning procedure is described hereinbelow.

Tobacco seeds were placed in the greenhouse and, after 4 weeks, total RNA was prepared from the seedlings. The total RNA was employed as template for the synthesis of double-stranded cDNA. The cDNA was filled up with Klenow fragment and phosphorylated with T4-polynucleotide kinase. Marathon adapters (5′-ctaatacgac tcactatagg gctcgagcgg ccgcccgggc aggt-3′/3′-cccg tcca-5′) were ligated onto the cDNA (Clontech, Advantage cDNA PCR Kit).

A fragment of the coding sequence of Nicotiana tabacum phytoene synthase was amplified with the aid of the PCR technique using the primers of the sequences 5′-tatgctaaga cgttttatct tggaac-3′ and 5′-ccatacaggc catctgctag c-3′. The amplified fragment was cloned into the bacterial vector pCR2.1 (Invitrogen) via TOPO TA-cloning. The sequence of the amplified fragment was determined by sequencing following the method of Sanger. Two different sequences were obtained and terned PSY1 and PSY2.

The 5′ terminus of the sequence of the Nicotiana tabacum phytoene synthase 1 was amplified with the aid of the PCR technique using the primers of the sequences 5′-ccatcgacta gctcatccgt tctcctgcac c-3′ and 5 ′-ccatcctaat acgactcacta tagggc-3′.

The 5′ terminus of the sequence of the Nicotiana tabacunz phytoene synthase 2 was amplified with the aid of the PCR technique using the primers of the sequences 5′-aagccggtct tcccacctat ctaaggcttg g-3′ and 5′-ccatcctaat acgactcacta tagggc-3′.

The 3′ termini of the sequence of Nicotiana tabacum phytoene synthases 1 and 2 were amplified with the aid of the PCR technique using the primers of the sequences 5′-agtaggactg atgagtgttc cagttatggg tattgcacc-3 ′ and 5′-ccatcctaat acgactcacta tagggc-3′.

The amplified fragments were cloned in the bacterial vector pCR2.1 (Invitrogen) via TOPO TA-cloning. The sequences of the amplified fragments were determined by sequencing following the method of Sanger.

The transcribed sequence of the Nicotiana tabacum phytoene synthase 1 was amplified with the aid of the PCR technique using the primers of the sequences 5′-agaaacccag aaagaacaac aggttttg-3′ and 5′-ctcacttgag ggtttgatga gtgtgg-3′. The amplified fragment was cloned into the bacterial vector pCR2.1 (Invitrogen) via TOPO TA cloning. The sequence of the amplified fragment was determined by sequencing following the method of Sanger. The sequence was termed PSY1.

The coding sequence of the Nicotiana tabacum phytoene synthase 1 was amplified with the aid of the PCR technique using the primers of the sequences 5′-ttcccgggtt gtttcatgag catg-3′ and 5′-ttcccgggtc attcatgtct ttgc-3′. The amplified fragment was recut with the restriction endonuclease Xma I. The resulting XmaI PSY1 fragment was ligated into the linearized and dephosphorylated vector pSS. The resulting constructs pSS-PSY1 were checked for the orientation of the transgene by restriction mapping.

The coding sequence of the Nicotiana tabacum phytoene synthase 2 was amplified with the aid of the PCR technique using the primers of the sequences 5′-atgaattctg ttcaaaatgt ctgttgcc-3′ and 5′-atgaattcct gatgtctatg ccttagctag ag-3′. The amplified fragment was recut with the restriction endonuclease EcoRI. The resulting EcoRI PSY2 fragment was ligated into the linearized and dephosphorylated vector pSS. The resulting constructs pSS-PSY2 were checked for the orientation of the transgene by restriction mapping.

### Example 2

### Cloning Nicotiana Tabacum Zeta-Carotene Desaturase

Tobacco seeds were placed in the greenhouse and, after 4 weeks, total RNA was prepared from the seedlings. The total RNA was employed as template for the synthesis of double-stranded cDNA. The cDNA was filled up with Klenow fragment and phosphorylated with T4-polynucleotide kinase. Marathon adapters (5′-ctaatacgac tcactatagg gctcgagcgg ccgcccgggc aggt-3′/3′-cccg tcca-5′) were ligated onto the cDNA (Clontech, Advantage cDNA PCR Kit).

A fragment of the coding sequence of Nicotiana tabacum zeta-carotene desaturase was amplified with the aid of the PCR technique using the primers of the sequences 5′-gagctggact tgcaggcatg tcg-3′and 5′-aactggaaga attcgcggcc gcaggaattt tttttttttt ttttt-3′. The amplified fragment was cloned into the bacterial vector pCR2.1 (Invitrogen) via TOPO TA cloning. The sequence of the amplified fragment was determined by sequencing following the method of Sanger.

The 5′ terminus of the sequence of the Nicotiana tabacum zeta-carotene desaturase was amplified with the aid of the PCR technique using the primers of the sequences 5′-tccacctcat gtccttgatc caagagctcc-3′ and 5′-ccatcctaat acgactcacta tagggc-3′. The amplified fragment was cloned into the bacterial vector pCR2.1 (Invitrogen) via TOPO TA cloning. The sequence of the amplified fragment was determined by sequencing following the method of Sanger.

The transcribed sequence of the Nicotiana tabacum zeta-carotene desaturase was amplified with the aid of the PCR technique using the primers of the sequences 5′-ctggcatctt acatctgcca aatttcc-3′ and 5′-tcttctcaat gaatgatgag caatacgatc c-3′. The amplified fragment was cloned into the bacterial vector pCR2.1 (Invitrogen) via TOPO TA cloning. The sequence of the amplified fragment was determined by sequencing following the method of Sanger. This gave the sequence SEQ ID NO. 1.

The coding sequence of the Nicotiana tabacum zeta-carotene desaturase was amplified with the aid of the PCR technique using the primers of the sequences SEQ ID NO. 3 and SEQ ID NO. 4. The amplified fragment was recut with the restriction endonuclease Xma I. The resulting XmaI ZDS fragment was ligated into the linearized and dephosphorylated vector pSS. The resulting constructs pSS-ZDS were checked for the orientation of the transgene by restriction mapping.

The found open reading frame for ZDS encodes a protein of 588 amino acids corresponding to ˜65 kDa. The homology with the known Capsicum anuum ZDS at amino acid level is 95%. Accordingly, this is the tobacco plastid ZDS.

### Example 3

### Cloning the Nicotiana Tabacum SR1 Phytoene Desaturase

Tobacco seeds were placed in the greenhouse and, after 4 weeks, total RNA was prepared from the seedlings. The total RNA was employed as template for the synthesis of single-stranded cDNA (Pharmacia, 1^{st} strand cDNA Synthesis Kit).

The coding sequence of the Nicotiana tabacum phytoene desaturase was amplified with the aid of the PCR technique using the primers of the sequences SEQ ID NO: 9 and SEQ ID NO: 10. The amplified fragment was recut with the restriction endonuclease Xma I. The resulting XmaI PDS fragment was ligated into the linearized and dephosphorylated vector pSS. The resulting constructs pSS-PDS were checked for the orientation of the transgene by restriction mapping.

### Example 4

### Southern and Northern Blots

To further characterize the tobacco PSY genes, a Southern blot was carried out. Genomic tobacco DNA was prepared and cut with various restriction endonucleases. The DNA was blotted onto nitrocellulose and hybridized with radiolabelled probe PSY1. In each case, 3 or 4 bands can be discerned. To obtain an indication to the functionality of the two or, if appropriate, more PSY genes in tobacco, tobacco seeds were placed in the greenhouse. After 2, 4 and 6 weeks, some of the plant material was harvested and frozen in liquid nitrogen. In parallel, petals of adult tobacco plants were harvested and treated in the same manner. mRNA preparations were carried out with this material. The mRNA was transferred to nitrocellulose (slot blot) and hybridized separately with radioactive PSY1 and PSY2 probes. In total, the two genes show highly different transcription levels. However, independently of the developmental stage, the ratio between the two genes remains constant.

### Example 5

### Switching Off the Genes Encoding Enzymes of the Carotenoid Biosynthetic Pathway In Tobacco

Primers for the amplification of the complete coding sequences were defined using the PSY, PDS and ZDS sequences obtained and the known sequence of tobacco LCY. PCR reactions were carried out with these primers. The amplificates were cloned into the bacterial vector pCR2.1 by TOPO TA cloning (Invitrogen). The identity of the insertions was checked by sequencing. Primers for the amplification with Xma I-restriction cleavage sites on both sides were defined for all genes. PCR reactions were carried out with these primers. The constructs of the genes in vector pCR2.1 were used as templates. The resulting fragments were cut with the restriction endonuclease Xma I. The binary vector pSS was also cut with Xma I and dephosphorylated with calf thymus alkaline phosphatase. The cut genes were ligated into vector pSS. The sense or antisense orientation of the genes in the constructs obtained was checked by restriction analyses. Several clones were selected, and the transitions between pSS and gene were additionally checked by sequencing. The selected constructs and the blank vector pSS (control) were transformed into competent S17.1 cells. The plasmids were transferred to Agrobacterium tumefaciens pMP90RK by conjugation. The agrobacterial cultures were checked for the presence of the plasmids by PCR. Tobacco plants were transformed with the pSS constructs in two different approaches. Protoplasts were isolated from 4-week-old Nicotiana tabacum SR1 shoot cultures. The protoplasts were transformed by coculture with the agrobacteria. The agrobacteria employed for coculture contained the gene constructs in sense and antisense orientation. Calli were grown from transformed protoplasts under suitable selection pressure. 70 calli were regenerated per construct and control. In parallel, leaf disc transformations were carried out with all constructs.

Following the transfer of regenerated shoots to canamycin-containing medium, the remaining calli of each individual construct were combined. mRNA was prepared from this material and transferred in 4 identical copies to a nitrocellulose membrane in the form of slot blots. In each case one copy was hybridized with one radioactive probe each of PSY1, PDS, ZDS and LCY. In all samples, strong signals appeared for the transcripts of the particular genes which had been transferred into the plants. The activity of the doubled 35S promoter is thus proven.

The regenerated shoots were rooted in sterile culture. The tip was transferred to fresh medium and the shoot was cut back. After the tip had rooted, the shoot, which had sprouted again, was transferred from the sterile culture in the greenhouse into soil. The rooted tip remained in sterile culture and was transplanted at regular intervals to fresh medium.

After 5-6 weeks, one leaf was cut off from the transgenic greenhouse plants at a level of approximately ⅔ of the plant. 0.5 g of this leaf was comminuted in a Potter together with a spatula-tip full of magnesium oxide and 3 ml of acetone. The digest was filtered, and xantophyll, carotenoid and chlorophyll content was determined via HPLC. The remaining leaf material was immediately frozen in liquid nitrogen and comminuted in a mortar. mRNA preparations for slot blots were carried out with this material.

### Example 6

### Phenotypic Analysis of the Transgenic Plants

Both in sterile culture and in the greenhouse, the transgenic plants with phytoene synthase in sense orientation show phenotypic effects, some of which were pronounced. The effect on leaf morphology is pronounced: some of the young leaves show a somewhat orangey-yellow coloration which, however, disappears with increasing age, while older leaves show very irregular pale green pigmentation. In addition, the leaves are succulent, hirsute and very firm, and their margins roll inwardly from the sides. The development of the plants in total is greatly retarded in comparison with control plants. The flowers of some plants which are particularly heavily affected are virtually colourless and do not show the pink coloration of the petals, which the control plants exhibit. The seed capsules are clearly orange instead of green.

Measurement of the carotenoid, xanthophyll and chlorophyll contents of these plants shows the accumulation of small amounts of phytoene and a marked increase in the β-carotene content. Phytoene as direct product of phytoene synthase cannot be detected in control plants under the prevailing conditions. Moreover, transgenic plants show up to 40% reduction in the chlorophyll content. This can be attributed to the redirection of geranylgeranyl pyrophosphate from phytol synthesis towards carotenoid synthesis, which, as a consequence, leads to reduced chlorophyll synthesis.

Transgenic plants with phytoene desaturase in antisense orientation show leaf pigmentation effects in the greenhouse. The leaves have white veins, and the leaf tips are completely white in some cases. The seed capsules of one line were also completely white. Measurement of the carotenoid contents of the lines in question show very high accumulation of phytoene in approximately the order of magnitude which is found in tobacco plants treated with norflurazon. The xanthophyll and chlorophyll contents are reduced. Seeds of a selfed plant of one of the lines in question were sown and were germinated under selection conditions. These seedlings showed segregation into 3 phenotypes: selected plants, which only have white cotyledones, and green plants and white plants which grew beyond the primary leaf stage. The white seedlings were homozygote transgenic plants. After transfer into the greenhouse, all white plants died within a week, while the green plants show the above-described phenotype of the adult heterozygote plants. Profiling showed very high accumulation of phytoene in the case of the green seedlings. No β-carotene and only small amounts of xanthophylls were detected in the white seedlings.

References

Altschul, S. F., Madden, T. L., Schaffer, A. A., Zhang, J. Z.; Miller W. and Lipman, D. J. 1997. Gapped BLAST und PSI-BLAST generation of protein database search programs. Nucleic Acids Res. 25: 3389-3402

Lichtenthaler, H. K., Schwender, J., Disch, A., Rohmer, M. Biosynthesis of isoprenoids in higher plant chloroplasts proceeds via a mevalonate-independent pathway. FEBS Letters (1997) 400, 271-274.

Pecker, I., Chamovitz, D., Linden, H., Sandmann, G., Hirschberg, J. A single poly-peptide catalyzing the conversion of phytoene to -carotene is transcriptionally regulated during tomato fruit ripening. Plant Molecular Biology (1996) 30, 807-819.

Bonk, M., Hoffmann, B., Lintig, von, J., Schledz, M., Al-Babili, S., Hobeika, E., Kleinig, H., Beyer, P. Chloroplast import of four carotenoid biosynthetic enzymes in vitro reveals differential fates prior to membrane binding and oligomeric assembly. Eur. J. Biochem. (1997) 247, 942-950.

Dogbo, O., Laferrière, A., d'Harlingue, A., Camara, B. Carotenoid biosynthesis: Isolation and characterization of a bifunctional enzyme catalyzing the synthesis of phytoene. PNAS (1988) 85, 7054-7058.

Fire, A., Xu, S. Montgomery, M. K., Kostas, S. A., Driver, S. E., Mello, C. C. Potent and specific genetic interference by double-stranded RNA in Caenorhabditis elegans. Nature (1998) 391, 806-811.

Fray, R. G., Wallace, A., Fraser, P. D., Valero, D., Hedden, P., Bramley, P. M., Grierson, D. Constitutive expression of a fruit phytoene synthase gene in transgenic tomatoes causes dwarfism by redirecting metabolites from the gibberellin pathway. Plant J. (1995) 8 (5), 693-701.

Lintig, von, J, Welsch, R., Bonk, M., Giuliano, G., Batschauer, A., Kleinig, H. Light-dependant regulation of carotenoid biosynthesis occurs at the level of phytoene synthase expression and is mediated by phytochrome in Sinapis alba and Arabidopsis thaliana seedlings. Plant J. (1997) 12(3), 625-634.

Lottspeich, F., Zorbas H. (Ed.) 1998. Bioanalytik [Bioanalytics]. Spektrum Akademischer Verlag, Heidelberg, Berlin.

Minet, M., Dufour, M. -E. and Lacroute, F. 1992. Complementation of Saccharomyces cerevisiae auxotrophic mutants by Arabidopsis thaliana cDNAs. Plant J. 2: 417-422.

Neudert, U., Martinez, I. M., Fraser, P. D., Sandmann, G. Expression of an active phytoene synthase from Erwinia uredovora and biochemical properties of the enzyme. Biochimics et Biophysica Acta (1998) 1392, 51-58.

Norris, S. R., Barrette, T., DellaPenna, D. Genetic dissection of carotenoid synthesis in Arabidopsis defines plastoquinone as an essential component of phytoene desaturation. Plant Cell (1995) 7, 2139-2149.

Wetzel, C. M., Rodermel, S. R. Regulation of phytoene desaturase expression is independant of leaf pigment content in Arabidopsis thaliana. Plant Molecular Biology (1998) 37, 1045-1053.

Corona, V., Aracri, B., Kosturkova, G., Bartley, G. E., Pitto, L., Giorgetti; L. Scolnik, P. A., Giuliano, G. Regulation of a carotenoid biosynthesis gene promoter during plant development. The Plant J. (1996) 9(4), 505-512.

Trebst, A., Depka, B. Role of carotene in the rapid turnover and assembly of photo-system II in Chlamydomonas reinhardtii. FEBS Letters (1997) 400, 359-362.

Cunningham, F. X., Pogson, B., Sun, Z., McDonald, K. A., DellaPenna, D., Gantt, E. Functional analysis of the β- and ε-lycopene cyclase enzymes of Arabidopsis reveals a mechanism for control of cyclic carotenoid formation. Plant Cell, 8: 1613-1626, 1996.

Explanations to the Sequence Listing and to the Figures

SEQ ID NO. 1

DNA sequence encoding a Nicotiana tabacum phytoene synthase. The amino acid sequence encoded by the DNA is stated.

SEQ ID NO. 2

Amino acid sequence of a polypeptide with the activity of Nicotiana tabacum phytoene synthase.

SEQ ID NO. 3

DNA sequence encoding a Nicotiana tabacum phytoene synthase. The amino acid sequence encoded by the DNA is stated.

SEQ ID NO. 4

Amino acid sequence of a polypeptide with the activity of Nicotiana tabacum phytoene synthase.

SEQ ID NO: 5

DNA sequence encoding a Nicotiana tabacum zeta-carotene desaturase. The amino acid sequence encoded by the DNA is stated.

SEQ ID NO: 6

Amino acid sequence of a polypeptide with the activity of Nicotiana tabacum zeta-carotene desaturase.

SEQ ID NO: 7

Oligonucleotide for amplifying the Nicotiana tabacum zeta-carotene desaturase by means of the PCR technique.

SEQ ID NO: 8

Oligonucleotide for amplifying the Nicotiana tabacum zeta-carotene desaturase by means of the PCR technique.

SEQ ID NO: 9

Oligonucleotide for amplifying the Nicotiana tabacum phytoene desaturase by means of the PCR technique.

SEQ ID NO: 10

Oligonucleotide for amplifying the Nicotiana tabacum phytoene desaturase by means of the PCR technique.

FIG. 1

Carotenoid biosynthesis in the plastids of plants; abbreviations: PSY: phytoene synthase; PDS: phytoene desaturase; ZDS: zeta-carotene desaturase; LCY: lycopene β-cyclase.

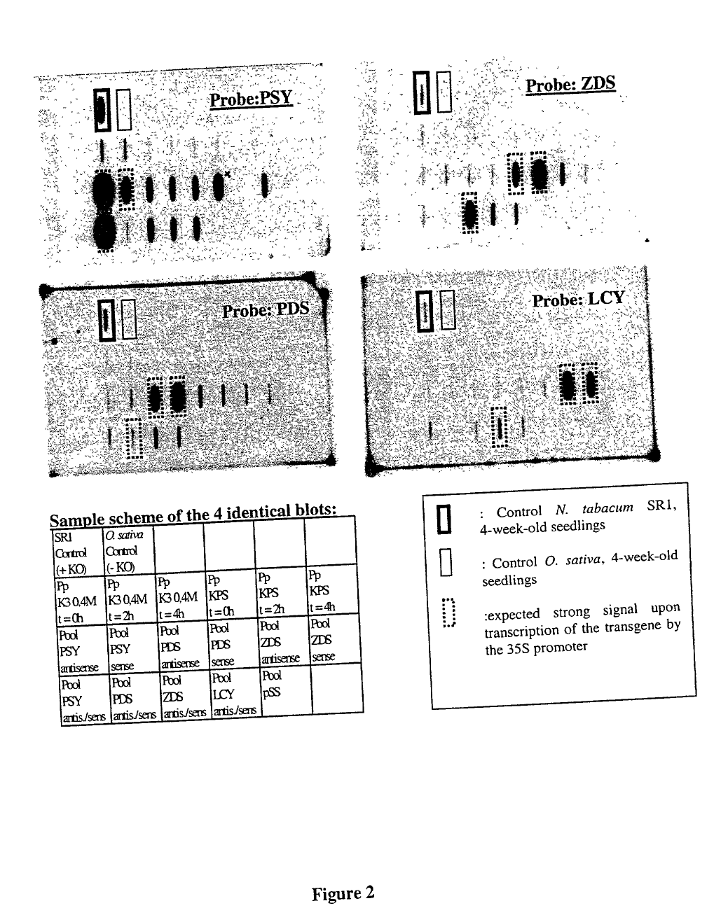

FIG. 2

Checking transcript accumulation in transgenic calli: following the transfer of regenerated shoots, the remaining calli of one transformation were combined. mRNA was prepared from these calli mixtures. In each case ˜500 ng of the mRNA were blotted onto a nitrocellulose membrane as shown in the sample scheme. In each case one of the membranes was hybridized with a radioactive probe of the four different genes. mRNAs of 4-week-old tobacco seedlings and of 4-week-old rice seedlings were used as controls. Abbreviations: Pp: protoplasts, K3 0.4M: K3 0.4M medium, KPS: KPS medium.

1. Nucleic acids which encode polypeptides from tobacco with the bioactivity of a zeta-carotene desaturase, which comprises the amino acid sequence of SEQ ID NO: 6.

2. Nucleic acids according to claim 1, characterized in that they encode polypeptides with the amino acid sequence of SEQ ID NO: 6.

3. Nucleic acids according to claim 1 or 2, characterized in that they are single-stranded or double-stranded DNA or RNA.

4. Nucleic acids according to claim 3, characterized in that they are fragments of genomic DNA or cDNA.

5. Nucleic acids according to one of claims 1 to 4, characterized in that they are derived from tobacco plants.

6. Nucleic acids according to one of claims 1 to 5, comprising a sequence selected from amongst
(a) the sequence of SEQ ID NO: 5,
(b) sequences encoding a polypeptide which comprises the amino acid sequence of SEQ ID NO: 6,
(c) part sequences of the sequences defined under (a) or (b) which are at least 14 base pairs in length,
(d) sequences which hybridize with the sequences defined under (a), (b) or (c),
(e) sequences which are complementary to the sequences defined under (a), (b) or (c), and
(f) sequences which, owing to the degeneracy of the genetic code, encode the same amino acid sequence as the sequences defined under (a) to (c).

7. Regulatory region which naturally controls, in plant cells, in particular in tobacco plants, the transcription of a nucleic acid according to one of claims 1 to 6.

8. DNA construct comprising a nucleic acid according to one of claims 1 to 6 and a heterologous promoter.

9. Vector comprising a nucleic acid according to one of claims 1 to 6, a regulatory region according to claim 7 or a DNA construct according to claim 8.

10. Vector according to claim 9, characterized in that the nucleic acid is linked functionally to regulatory sequences which ensure the expression of the nucleic acid in pro- or eukaryotic cells.

11. Host cell containing a nucleic acid according to one of claims 1 to 6, a DNA construct according to claim 8 or a vector according to claim 9 or 10.

12. Host cell according to claim 11, characterized in that it is a prokaryotic cell, in particular E. Coli.

13. Host cell according to claim 11, characterized in that it is a eukaryotic cell, in particular a yeast cell, insect cell, mammalian cell or plant cell.

14. Polypeptide with the bioactivity of a phytoene synthase which is encoded by a nucleic acid of SEQ ID NO: 1 or SEQ ID NO: 3, comprising an amino acid sequence of SEQ ID NO: 2 or SEQ ID NO: 4.

15. Polypeptide with the bioactivity of a zeta-carotene desaturase which is encoded by a nucleic acid according to one of claims 1 to 6.

16. Antibody which binds specifically to a polypeptide of claim 14.

17. Antibody which binds specifically to a polypeptide of claim 15.

18. Process for generating a nucleic acid according to one of claims 1 to 6, comprising the following steps:
(a) complete chemical synthesis carried out in a manner known per se or
(b) chemical synthesis of oligonucleotides, labelling the oligonucleotides, hybridizing the oligonucleotides with DNA of a genomic or cDNA library which has been generated starting from genomic DNA or mRNA of plant cells, selection of positive clones and isolation of the hybridizing DNA from positive clones, or
(c) chemical synthesis of oligonucleotides and amplification of the target DNA by means of PCR.

19. Process for generating a polypeptide according to claim 14 or 15, comprising
(a) culturing a host cell according to one of claims 11 to 13 under conditions which ensure the expression of the nucleic acid according to one of claims 1 to 6, or
(b) expressing a nucleic acid according to one of claims 1 to 6 in an in-vitro system, and
(c) obtaining the polypeptide from the cell, the culture medium or the in-vitro system.

20. Method of finding a chemical compound which binds to a polypeptide according to claim 14 and/or 15 or a polypeptide with the bioactivity of a phytoene desaturase, comprising the following steps:
(a) contacting a host cell according to one of claims 11 to 13, a polypeptide according to claim 14 or 15 or a polypeptide with the bioactivity of a phytoene desaturase with a chemical compound or a mixture of chemical compounds under conditions which permit the interaction of a chemical compound with the polypeptide, and
(b) determining the chemical compound which binds specifically to the polypeptide.

21. Method of finding a compound which modifies the expression of polypeptides according to claim 14 or 15 or a polypeptide with the bioactivity of a phytoene desaturase, comprising the following steps:
(a) contacting a host cell according to one of claims 11 to 13 with a chemical compound or a mixture of chemical compounds,
(b) determining the polypeptide concentration, and
(c) determining the compound which specifically influences the expression of the polypeptide.

22. Use of a nucleic acid according to one of claims 1 to 6, of a DNA construct according to claim 8, of a vector according to claim 9 or 10, of a host cell according to one of claims 11 to 13, of a polypeptide according to claim 14 or 15 or of a polypeptide with the bioactivity of a phytoene desaturase or of an antibody according to claim 16 or 17 for finding new herbicidal active substances.

23. Use of a modulator of a polypeptide according to claim 14 or 15 or of a polypeptide with the bioactivity of a phytoene desaturase as plant growth regulator or herbicide.

24. Use of a nucleic acid according to one of claims 1 to 6, of a DNA construct according to claim 8 or a vector according to claim 9 or 10 for generating transgenic plants.

25. Transgenic plants, parts of plants, protoplasts, plant tissues or plant propagation materials, characterized in that the intracellular concentration of a polypeptide according to claim 16 or 17 is increased or reduced in comparison with the corresponding wild-type cells after introducing a nucleic acid according to one of claims 1 to 6, a DNA construct according to claim 8 or a vector according to claim 9.

26. Plants, parts of plants, protoplasts, plant tissues or plant propagation materials, characterized in that they contain a polypeptide according to claim 14 or 15 whose bioactivity or expression pattern is modified in comparison with the corresponding endogenous polypeptides.

27. Method of generating plants, parts of plants, protoplasts, plant tissues or plant propagation materials according to claim 25, characterized in that a nucleic acid according to one of claims 1 to 6 or a regulatory region according to claim 7 is modified by endogenous mutagenesis.
